(19) United States
(12) Patent Application Publication
(10) Pub. No.: US 2022/0104731 A1
(43) Pub. Date: Apr. 7, 2022

(54) IN-VIVO GLUCOSE SPECIFIC SENSOR

(71) Applicant: Zense-Life Inc., Carlsbad, CA (US)

(72) Inventors: Huashi Zhang, San Juan Capistrano, CA (US); Robert James Boock, Carlsbad, CA (US); Michael Wheelock, San Clemente, CA (US); Mark Wu, San Diego, CA (US); Qinyi Yan, San Diego, CA (US); Yubin Huang, Vista, CA (US); Steven Soto, San Marcos, CA (US); Jessie Haskamp, San Diego, CA (US)

(73) Assignee: Zense-Life Inc., Carlsbad, CA (US)

(21) Appl. No.: 17/449,380

(22) Filed: Sep. 29, 2021

Related U.S. Application Data
(60) Provisional application No. 63/087,566, filed on Oct. 5, 2020.

(51) Int. Cl. A61B 5/1486; A61B 5/145

(52) U.S. Cl. CPC A61B 5/1486; A61B 5/14532; A61B 5/14503; A61B 2562/164; A61B 2562/125; A61B 2562/227; A61B 5/1451

(57) ABSTRACT

A glucose-specific sensor has a glucose limiting layer (GLL), an enzyme layer and an interference layer. The GLL comprises polyurethane with a molecular weight greater than 100,000 Daltons that is physically crosslinked with a water-soluble polymer having a molecular weight greater than 100,000 Daltons. The interference layer has a polymer formed from pyrrole, phenylenediamine (PDA), aminophenol, aniline, or combinations thereof. Methods for making a glucose-specific sensor include mixing a monomer with a solvent to form a monomer solution, applying the monomer solution to a substrate and electropolymerizing the monomer to form a polymer on the substrate. The polymer is an interference layer for the glucose-specific sensor. An enzyme layer is formed on the interference layer, and a glucose limiting layer is formed on the enzyme layer.

### RELATED APPLICATIONS

This application claims priority to U.S. Provisional Application No. 63/087,566 filed on Oct. 5, 2020, and entitled “In-Vivo Glucose Specific Sensor Having Simplified Calibration,” which is incorporated herein as if set forth in its entirety.

This application is related to U.S Provisional Application 63/037,072 filed Jun. 10, 2020, and entitled “Sterilizable Metabolic Analyte Sensor,” which is incorporated herein as if set forth in its entirety. This application is also related to U.S. patent application Ser. No. 16/375,875, filed Apr. 5, 2019 and entitled “An Enhanced Interference Membrane for a Working Electrode of a Continuous Biological Sensor”; which claims priority to (1) U.S. Provisional Application No. 62/653,821, filed Apr. 6, 2018, and entitled “Continuous Glucose Monitoring Device”; (2) U.S. Provisional Application No. 62/796,832, filed Jan. 25, 2019, and entitled “Carbon Working Electrode for a Continuous Biological Sensor”; and (3) U.S. Provisional Application No. 62/796,842, filed Jan. 25, 2019, and entitled “Enhanced Membrane Layers for the Working Electrode of a Continuous Biological Sensor”; each of which is incorporated herein as if set forth in their entirety.

### BACKGROUND

Medical patients often have diseases or conditions that require the measurement and reporting of biological conditions. For example, if a patient has diabetes, it is important that the patient have an accurate understanding of the level of glucose in their blood. Traditionally, diabetes patients have monitored their glucose levels by sticking their finger with a small lancet, allowing a drop of blood to form, and then dipping a test strip into the blood. The test strip is positioned in a handheld monitor that performs an analysis on the blood and visually reports the measured glucose level to the patient. Based upon this reported level, the patient makes important decisions on what food to consume, or how much insulin to inject into their blood. Although it would be advantageous for the patient to check glucose levels many times throughout the day, many patients fail to adequately monitor their glucose levels due to the pain and inconvenience. As a result, the patient may eat improperly or inject either too much or too little insulin. Either way, the patient has a reduced quality of life and increased chance of doing permanent damage to their health and body. Diabetes is a devastating disease that if not properly controlled can lead to terrible physiological conditions such as kidney failure, skin ulcers, or bleeding in the eyes, and eventually blindness, and pain and the eventual amputation of limbs.

Regular and accurate monitoring of glucose levels is critical for diabetes patients. To facilitate such monitoring, continuous glucose monitoring (CGM) sensors are a type of device in which glucose is automatically measured from fluid sampled in an area just under the skin multiple times a day. CGM devices typically involve a small housing in which the electronics are located and which is adhered to the patient's skin to be worn for a period of time. A small needle within the device delivers the subcutaneous sensor which is often electrochemical. In this way, a patient may install a CGM on their body, and the CGM will provide automated and accurate glucose monitoring for many days without any action required from the patient or a caregiver. It will be understood that depending upon the patient's needs, continuous glucose monitoring may be performed at different intervals. For example, some continuous glucose monitors may be set or programmed to take multiple readings per minute, whereas in other cases the continuous glucose monitor can be programmed or set to take readings every hour or so. It will be understood that a continuous glucose monitor may sense and report readings at different intervals.

Continuous glucose monitoring is a complicated process, and it is known that glucose levels in the blood can significantly rise/increase or lower/decrease quickly, due to several causes. A single glucose measurement provides only a snapshot of the instantaneous level of glucose in a patient's body. Such a single measurement provides little information about how the patient's use of glucose is changing over time, or how the patient reacts to specific dosages of insulin. Accordingly, even a patient that is adhering to a strict schedule of strip testing will likely be making incorrect decisions as to diet, exercise, and insulin injection. Of course, this is exacerbated by a patient that is less consistent on performing their strip testing. To give the patient a more complete understanding of their diabetic condition and to get a better therapeutic result, some diabetic patients are now using continuous glucose monitoring.

A significant deficiency in known CGM sensors is that they exhibit substantial variability patient to patient, and even have sensitivity variability for a given patient over time. More particularly, the CGM sensors have variations in sensitivity to blood glucose concentrations, and so must be locally calibrated by each patient prior to use, and then recalibrated over time for a particular user. Unfortunately, the local calibration processes require the patient pricking their finger and obtaining a blood glucose reading using a standard strip monitor. Not only is local calibration inconvenient, time consuming, and prone to error, it can also be painful such that a patient may delay or avoid local calibration, thereby defeating any possible benefit from the CGM system.

Electrochemical glucose sensors operate by using electrodes which typically detect an amperometric signal caused by oxidation of enzymes during conversion of glucose to gluconolactone. The amperometric signal can then be correlated to a glucose concentration. Two-electrode (also referred to as two-pole) designs use a working electrode and a reference electrode, where the reference electrode provides a reference against which the working electrode is biased. The reference electrodes essentially complete the electron flow in the electrochemical circuit. Three-electrode (or three-pole) designs have a working electrode, a reference electrode and a counter electrode. The counter electrode replenishes ionic loss at the reference electrode and is part of an ionic circuit.

Known CGM systems typically use a working wire that uses a core of tantalum on which a thin layer of platinum is deposited. Tantalum is a relatively stiff material, so is able to be pressed into the skin without bending, although an introducer needle may be used to facilitate insertion. Further, tantalum is inexpensive as compared to platinum, which makes for an economical working wire. As is well known, an enzyme layer is deposited over the platinum layer, which is able to accept oxygen molecules and glucose molecules from the user's blood. The key chemical processes for glucose detection occur within the enzyme membrane. Typically, the enzyme membrane has one or more glucose oxidase enzymes (GOx) dispersed within the enzyme membrane. When a molecule of glucose and a molecule of oxygen (O₂) are combined in the presence of the glucose oxidase, a molecule of gluconate and a molecule of hydrogen peroxide (H₂O₂) are formed. In one construction, the platinum surface facilitates a reaction wherein the hydrogen peroxide reacts to produce water and hydrogen ions, and two electrons are generated. The electrons are drawn into the platinum by a bias voltage placed across the platinum wire and a reference electrode. In this way, the magnitude of the electrical current flowing in the platinum is intended to be related to the number of hydrogen peroxide reactions, which is intended to be related to the number of glucose molecules oxidized. A measurement of the electrical current on the platinum wire can thereby be associated with a particular level of glucose in the patient's blood or interstitial fluid (ISF).

The working wire is then associated with a reference electrode, and in some cases one or more counter electrodes, which form the CGM sensor. In operation, the CGM sensor is coupled to and cooperates with electronics in a small housing in which, for example, a processor, memory, a wireless radio, and a power supply are located. The CGM sensor typically has a disposable applicator device that uses a small introducer needle to deliver the CGM sensor subcutaneously into the patient. Once the CGM sensor is in place, the applicator is discarded, and the electronics housing is attached to the sensor. Although the electronics housing is reusable and may be used for extended periods, the CGM sensor and applicator need to be replaced quite often, usually every few days.

### SUMMARY

In embodiments, a glucose-specific sensor for in-vivo use in a patient includes a glucose limiting layer comprising a polyurethane with a molecular weight greater than 100,000 Daltons that is physically crosslinked with a water-soluble polymer having a molecular weight greater than 100,000 Daltons. The sensor also includes an enzyme layer comprising glucose oxidase (GOx) for reacting with in-vivo glucose in body fluid from the patient to generate hydrogen peroxide (H₂O₂). An interference layer comprises a polymer formed from pyrrole, phenylenediamine (PDA), aminophenol, aniline, or combinations thereof, where the enzyme layer is between the interference layer and the glucose limiting layer. A substrate having a conductive surface is adjacent the interference layer, for carrying an electric current generated in response to an in-vivo glucose concentration of the patient.

In embodiments, a glucose-specific sensor for in-vivo use in a patient includes a glucose limiting layer comprising a polyurethane with a molecular weight greater than 100,000 Daltons that is physically crosslinked with a water-soluble polymer. An enzyme layer comprises glucose oxidase (GOx) for reacting with in-vivo glucose in body fluid from the patient to generate hydrogen peroxide (H₂O₂). An interference layer comprises pyrrole and phenylenediamine (PDA), where the enzyme layer is between the interference layer and the glucose limiting layer. A substrate has a conductive surface adjacent the interference layer for carrying an electric current in response to an in-vivo glucose concentration of the patient.

In embodiments, methods for making a glucose-specific sensor for in-vivo use in a patient include mixing a monomer with a solvent to form a monomer solution and applying the monomer solution to a substrate having a conductive surface. The monomer is electropolymerized to form a polymer on the substrate, the polymer being an interference layer for the glucose-specific sensor. An enzyme layer is formed on the interference layer, and a glucose limiting layer is formed on the enzyme layer.

### BRIEF DESCRIPTION OF THE DRAWINGS

Objects and advantages of the present disclosure will become apparent upon reading the following detailed description and upon referring to the drawings and claims.

FIG. 1A is a flowchart of a prior art calibration process.

FIG. 1B is a sensitivity chart reflecting a prior art calibration process.

FIG. 2 is a not-to-scale radial cross-sectional diagram of a working wire for a glucose-specific sensor in accordance with some embodiments.

FIG. 3A is a not-to-scale longitudinal cross-sectional diagram of a glucose-specific sensor for a continuous glucose monitor in accordance with some embodiments.

FIG. 3B is a sensitivity chart reflecting a calibration process for a continuous glucose monitor in accordance with some embodiments.

FIG. 3C is a sensitivity chart reflecting a calibration process for a continuous glucose monitor in accordance with some embodiments.

FIG. 4 is a flowchart of a process for making and applying an interference layer for a glucose-specific sensor in accordance with some embodiments.

FIG. 5 is a flowchart of a process for making a working wire for a glucose-specific sensor with some embodiments.

FIG. 6 is a flowchart of a process for making a working wire for a glucose-specific sensor in accordance with some embodiments.

FIG. 7 is a flowchart of a process of using a glucose-specific sensor in accordance with some embodiments.

### DETAILED DESCRIPTION

In some embodiments, a glucose-specific sensor is constructed with a glucose-specific analyte sensor coupled to electronic operating circuitry. The glucose-specific analyte sensor has a set of membrane layers on (e.g., concentrically formed) an electrically conductive substrate (e.g., a platinum or platinum coated core). The set of membrane layers includes an interference membrane and an enzyme membrane selected for glucose. A glucose limiting membrane is also used. In the glucose-specific sensor, at least three membranes—glucose limiting, enzyme, and interference—cooperate and act together to nearly eliminate electroactive contaminant compounds from interfering with the current generated responsive to the presence of glucose. Because of the significant reduction of contaminant interference in the electric current generated by the glucose-specific sensor, the electric current can be attributed solely due to the presence of glucose. As a result, calibration variances between individuals or for one individual over time are greatly reduced, enabling the elimination of requiring an in-vivo calibration for the glucose-specific sensor by the patient.

In one example embodiment, a glucose specific sensor has a substrate with a conductive surface, an interference layer, an enzyme layer, and a glucose limiting layer. The glucose specific sensor is inserted in a patient to contact a patient's body fluid, such as blood or ISF. In this disclosure, references to glucose sensing in ISF shall also apply to glucose sensing in blood. The outer glucose limiting layer is constructed to pass a determined level of glucose from the blood or ISF to the enzyme layer, but effectively blocks a significant amount, such as over 95%, of the active electrochemical contaminants in the blood or ISF from the enzyme layer. The enzyme layer includes the GOx enzyme, which reacts with the glucose to generate H₂O₂. The interference layer, which is between the enzyme layer and the conductive surface, blocks nearly all of the active electrochemical contaminants in the enzyme layer from ever reaching the conductive surface, while freely passing the H₂O₂ to the conductive surface. The H₂O₂ reacts with the conductive surface, which is typically platinum, with the reaction generating free electrons that flow on the conductive surface to the sensor's electronics. The current generated from the H₂O₂ reaction is proportional to the concentration of glucose in the patient's blood or ISF, and is referred to as the “glucose current.” Although the glucose limiting layer has blocked over 95% of the active electrochemical contaminants, and most of the rest were blocked in the interference layer, it is possible that a small number of active electrochemical contaminants pass to the conductive surface where they react with the conductive surface to create a current referred to as the “contaminant current.” However, due to the effective blocking of the active electrochemical contaminants, the contaminant current is so small that it has no meaningful effect on calculating and communicating a blood glucose level. Depending on the specific construction of the glucose-specific sensor, the glucose current can be 100, 500, 1000 or even 10,000 times larger than the contaminant current. It is this incredibly high signal (glucose current) to noise (contaminant current) ratio that enables the total current flowing from the sensor to be attributed solely to the glucose current.

In a specific embodiment, the glucose limiting layer comprises polyurethane with a molecular weight (MW)>100,000 Daltons that is physically crosslinked with a water-soluble polymer having a molecular weight >100,000 Daltons. This construction was observed and tested to block more than 95% of the active electrochemical contaminants from passing into the enzyme layer, while still passing sufficient levels of glucose from the patient's blood into the enzyme layer. In this way, less than 5% of the active electrochemical contaminants reach the enzyme layer. Physically crosslinking means that the polymers are crosslinked through non-covalent bonding, such as hydrogen bonding or hydrophobic interaction between two polymers in the formulation. For example, in some embodiments physical crosslinking is in the form of hydrogen bonding between polyurethane and a water-soluble polymer. The interference layer is also configured to further block active electrochemical contaminants. In one example, the interference layer comprises pyrrole and phenylenediamine (PDA), where the PDA may be poly(p-phenylenediamine) (i.e., p-PDA) or poly(m-phenylenediamine) (i.e., mPDA). The pH of the solution for polymerizing the PDA is adjusted to tune the formation of the interference layer, which in turn determines the size of electroactive contaminants that are blocked by the interference layer. Test results in accordance with the present disclosure show that such an interference layer can block nearly all the active electrochemical contaminants from ever reaching the conductive surface of the substrate. This blocking of contaminants is achieved while at the same time making the interference layer sufficiently thin that it passes a high level of H₂O₂. By blocking nearly all the active electrochemical contaminants and passing an extremely high level of H₂O₂, any electric current generated at the conductive surface will be dominated by the electric current generated from the H₂O₂, and the electric current from the active electrochemical contaminants will be nearly zero, or at least so small that it is such a minor noise signal that it has no appreciable effect of the measurement of the glucose level.

Advantageously, when the glucose-specific sensor is in an in-vivo environment, its current raw response is linear to the in-vivo glucose concentration without an intercept (zero baseline). With the elimination of in-vivo baseline, the glucose-specific sensor significantly reduces the individual subject variances for the glucose-specific sensor, which leads to the elimination of the in-vivo SMBG calibration for the glucose-specific sensor by the patient. Further, due to the enhanced stability of the sensitivity response, the glucose-specific sensor does not have to be locally calibrated by a user during the entire useful life of the sensor. In this way, once the glucose-specific sensor has been calibrated in the factory, it does not ever need to be calibrated again. By eliminating the need for local calibration, the glucose-specific sensor is enabled to operate with simpler electronics and avoids the need for painful finger sticks.

The present disclosure relates to structures and processes for a glucose-specific analyte sensor, that is, the biological sensor is constructed to generate an electric current only due to the presence of glucose in a patient's body fluid, and is able to substantially eliminate the electrical interference from all contaminants. Further, the present devices and methods describe novel layers and processes for a CGM glucose-specific sensor that enable accurate operation without need for any finger stick calibration by the user. Importantly, this enables the glucose-specific sensor to be immediately usable in any human, thereby eliminating the need to calibrate to each individual user. Instead, all that is needed is a factory calibration, which provides a huge advantage in terms of ease of use compared to the traditional CGM device.

For a CGM sensor, typically the platinum layer is wrapped with an electrically insulating layer, and a band of the insulating layer is removed during manufacturing to expose a defined and limited portion of the conductive (e.g., platinum) wire, which exposes that region of the platinum to the enzyme layer. The removal of this band must be done very accurately and precisely, as this affects the overall electrical sensitivity of the sensor. As would be expected, accurately forming this band adds expense, complexity, and uncertainty to the manufacturing process.

Having direct contact between the enzyme layer and the platinum layer has disadvantages. First, the actual useful exposed area of an exposed portion of the platinum wire is substantially reduced by oxidation contamination, which also may lead to unpredictable and undesirable sensitivity results. In order to overcome this deficiency, the sensor must be subjected to sophisticated and ongoing calibration. Further, the bias voltage between the platinum wire and the reference electrode must be set relatively high, for example between 0.4-1.0 V. Such a high bias voltage is required to draw the electrons into the platinum wire, but also acts to attract contaminants from the blood or ISF into the sensor. These contaminants such as acetaminophen, ascorbic acid and uric acid interfere with the chemical reactions, leading to false and misleading glucose level readings.

Since these active contaminants are present at different levels in different patients, and at different levels in the same patient over time, conventional CGM sensors must be initially calibrated to each individual user, and multiple times for an individual user. Take for example a patient that has chronic pain and is on a daily regimen of acetaminophen as compared to a patient that is not taking any acetaminophen. If both patients have the same actual blood glucose, the patient taking the acetaminophen will generate a higher electrical current in the sensor due to the electrochemical reaction of the acetaminophen. Accordingly, if the sensors relied only on factory calibration, the CGM for the patient taking the acetaminophen would report a much higher blood glucose level as compared to the other patient. As this is completely unacceptable, each patient must calibrate their CGM with one or more finger-prick tests to calibrate the CGMs to the level of acetaminophen. Continuing the example, if the patient in pain improves and reduces the level of acetaminophen use, or the other patient is hurt and begins taking acetaminophen, both CGMs will show false readings, and will need to be locally recalibrated by each patient using finger-prick tests.

The presence of the active electrochemical contaminants, such as acetaminophen, ascorbic acid and uric acid cause the generation of an unwanted electrical signal or current, which adds to the electrical signal generated responsive to the presence of glucose. Thus, the resulting electrical signal received by the CGM's electronics has a component that is due to the glucose, and a component due to the presence of contaminants. Unless the CGM is able to accurately account for and remove the effects of the interference signal, the resulting glucose reading will be inaccurate and of little use to the patient. In order to compensate for the contaminants, conventional CGM devices require sophisticated calibration algorithms in the CGM electronics that rely on periodic comparison to results that a user gets from a finger stick blood glucose measurement. The finger stick process, which is more formally known as Self-Monitoring of Blood Glucose (SMBG) is the well-established process where a user sticks themselves with a lance and allows a drop of blood to be drawn into a housing, and a few seconds later a blood glucose level is displayed to the user. Because every person's blood, eating habits, and physiology are different, CGM devices must be calibrated individually to each person. To calibrate a conventional CGM, the user inserts the CGM and begins the continuous monitoring process. The user may notice that the CGM is giving readings far different from lab results, or the CGM unit itself may indicate that finger stick (SMBG) calibration is needed. The user waits until they believe their glucose level is stable and takes a finger stick reading. They then enter the finger stick reading into the CGM, for example by using a smartphone that is wirelessly connected to the CGM. The CGM then recalibrates its algorithms to compensate for the presence of contaminants. It is not unusual for a user to have to recalibrate the CGM multiple times in a two-week period as the sensitivity of the sensor changes over time.

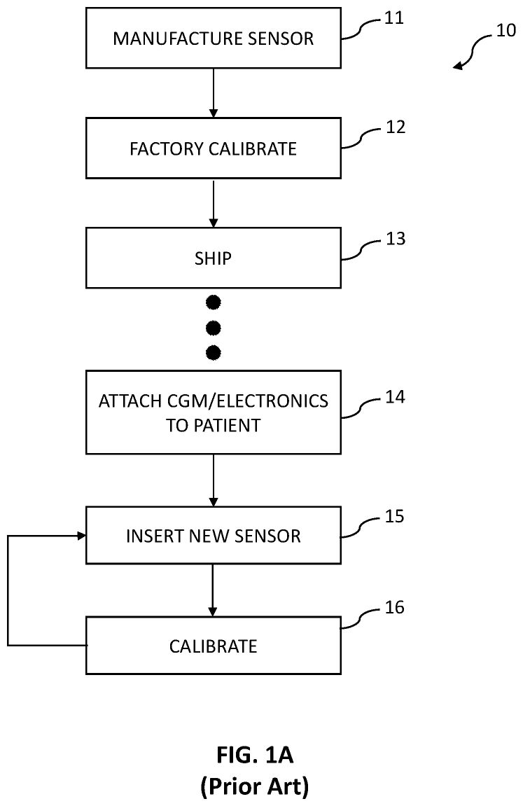

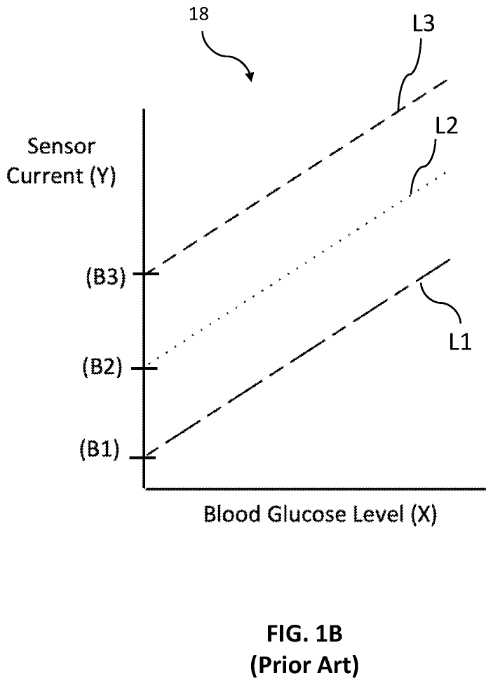

Referring now to the flowchart of FIG. 1A, a prior art method 10 of manufacturing and calibrating a biological sensor is illustrated. In step 11 the sensor is manufactured and then factory calibrated in step 12. The sensor is packaged and shipped in step 13 to a medical practitioner or to the user directly. In step 14 the user adheres the CGM and electronics to their body and inserts a new sensor subcutaneously in step 15. Then, from time to time the CGM electronics must be calibrated in step 16 by the user. This calibration of step 16 first has the user recognizing that the CGM reading has become inaccurate, either by an indication from the display of the CGM, or by comparison to a lab result or finger stick. Before beginning the calibration process, the user waits for a period of known blood glucose stability, does a finger stick (SMBG), and then enters the finger stick reading into the CGM. The CGM performs an internal calibration process and begins processing glucose information according to the new calibrated factors. As the subcutaneous sensors are only usable for a period of time, and the electronics are typically reusable, the user may insert a new sensor (step 15) from time to time. As illustrated by the loop of steps 15 and 16 in the flowchart of FIG. 1A, each sensor will typically be calibrated one or more times during its usable period.

The prior art CGM of FIG. 1A is a sensor that is inserted into the body subcutaneously to measure blood sugar levels in real time. The CGM sensor consists of two parts: a wire probe and electronic transmitter. The wire probe is inserted into the interstitial fluid of the body for glucose measuring. The electronics are connected to the probe and record signals from the probe, calculate glucose conversions, and transmit the data as needed. To use the CGM sensor, it must be first calibrated at the factory, and then again for the individual user. Due to contaminants that have reached the working wire, the electrical signal on the working wire's conductor includes electrical noise and currents from the contaminants. These noise and contaminant signals must be accounted for to obtain an accurate reading.

FIG. 1B shows a sensitivity chart 18 of the electrical response for a prior art CGM sensor. Chart 18 is a graph that has an X axis that represents the blood glucose level present in a user, which is typically measured in milligrams per deciliter (mg/dL). The Y-axis represents the amount of current flowing on the working wire (sensor current), which is typically measured in nanoamperes (nA). As illustrated in sensitivity chart 18, three user responses are shown by three different dashed lines L1, L2 and L3. These user responses may be from three different users or may be from the same user at different times. As can be seen, although each of the user responses is linear, each has a very different baseline—labeled as B1, B2, B3 for lines L1, L2, L3, respectively. This baseline is the sensor current at a blood glucose level of zero and represents the amount of the sensor current that is attributed to noise or contaminant interference. This noise/contamination must be accounted for in a user-specific calibration process as discussed above. As can be seen, the response for the sensor is generally linear and follows the algebraic equation of Y=AX+B where A (the slope of the line, rise over run) is the glucose sensitivity and B is the baseline. Generally speaking, value “A” represents how sensitive the sensor is towards glucose and value “B” represents how specific the sensor is towards glucose. Prior art CGM sensors typically have a significantly high in-vivo baseline, which is caused by the in-vivo interferent compounds, such as acetaminophen, ascorbic acid and uric acid.

Due to the noise and unwanted currents generated responsive to the active electrochemical contaminants, prior art CGM monitors must be calibrated to each individual user, and often times need continual calibration during the lifecycle of the sensor on a single user. This results in the need for more powerful processors, more memory, as well as uncertainty as to accuracy. Further, the local calibration process often requires a finger stick, which is counterproductive to the benefits that the CGM system provides.

A Working Wire for a Glucose-Specific Sensor

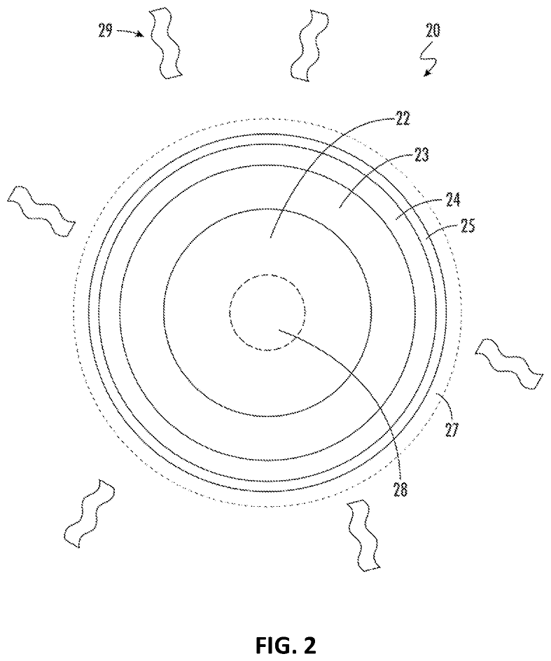

Referring now to FIG. 2, a working wire 20 for a continuous glucose-specific sensor is illustrated. The working wire 20 is constructed with a substrate 22, which may be, for example tantalum. It will be appreciated that other substrates may be used, such as a Cr—Co alloy as set forth in U.S. patent application Ser. No. 17/302,415 entitled “Working Wire for a Biological Sensor” and filed on May 3, 2021; or a plastic substrate with a carbon compound as set forth in in co-pending U.S. patent application Ser. No. 16/375,887 entitled “A Carbon Working Electrode for a Continuous Biological Sensor” and filed on Apr. 5, 2019; all of which are hereby incorporated by reference. It will be appreciated that other substrate materials may be used. In general, the substrate 22 has an electrically conductive surface (i.e., outer surface) that is a conductive material. The conductive surface may be a metal, and may include platinum, platinum/iridium alloy, platinum black, gold or alloys thereof, palladium or alloys thereof, nickel or alloys thereof, titanium and alloys thereof. The conductive surface may include carbon in different forms, such as one or more carbon allotropes including nanotubes, fullerenes, graphene and/or graphite. The conductive surface may also include a carbon material such as diamagnetic graphite, pyrolytic graphite, pyrolytic carbon, carbon black, carbon paste, or carbon ink. In the embodiment of FIG. 2, the substrate 22 has a continuous layer 23 which is an outer surface of the substrate that is an electrically conductive. In this embodiment, continuous layer 23 shall be described as platinum, although other conductive materials may be used as described throughout this disclosure. This platinum layer may be provided through an electroplating or depositing process, or in some cases may be formed using a drawn filled tube (DTF) process. It will be appreciated that other processes may be used to apply the platinum continuous layer 23.

The substrate 22, platinum continuous layer 23, an interference layer 24, an enzyme layer 25 and a glucose limiting layer 27 form key aspects of working wire 20. It will be understood that other layers may be added depending upon the particular biologic being tested for, and application-specific requirements. In some cases, the substrate 22 may have a core portion 28. For example, if the substrate 22 is made from tantalum, a core of titanium or titanium alloy may be provided to provide additional strength and straightness. Other substrate materials may use other materials for its core 28.

Interference layer 24 is applied over the platinum continuous layer 23. This interference layer, which will be fully described below, fully encases the platinum continuous layer 23, and is set between the platinum continuous layer 23 and the enzyme layer 25. This interference layer 24 is constructed to fully wrap the platinum, thereby protecting the platinum from further oxidation effects. The interference layer 24 is also constructed to substantially restrict the passage of larger contaminant molecules, such as acetaminophen, to reduce contaminants that can reach the platinum and skew the electrical signal results. Further, the interference layer 24 is able to pass a controlled level of hydrogen peroxide (H₂O₂) from the enzyme layer to the platinum layer, thereby increasing sensitivity, stability and accuracy. Enzyme layer 25 is then applied over the interference layer 24, and finally glucose limiting layer 27 is layered on top of the enzyme layer 25. As will be discussed fully below, the glucose limiting layer 27 is constructed and formulated to block or reject a significant amount, such as over 95%, of the active electrochemical contaminants present in the patient's blood, while still passing sufficient glucose into the enzyme layer. The working wire 20 must be able to withstand exposure to sterilization 29 which may be, for example, ethylene oxide (EtO) gas.

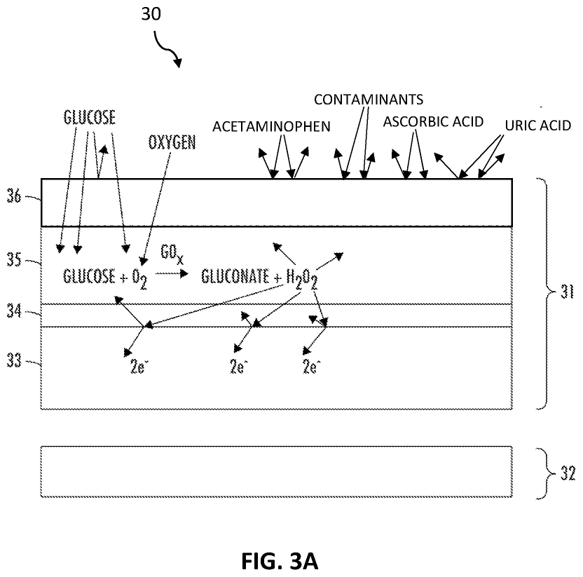

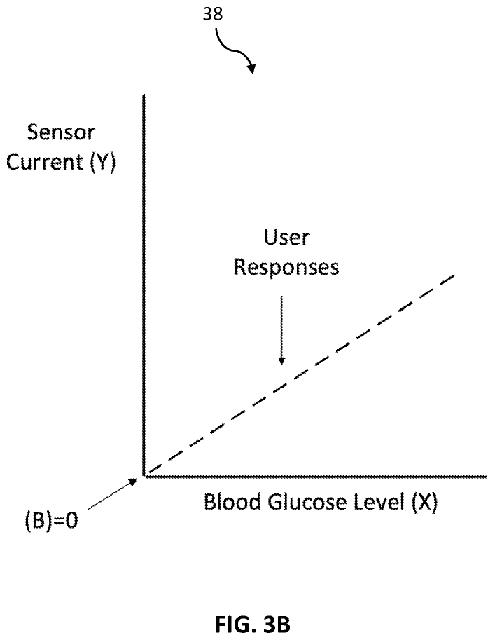

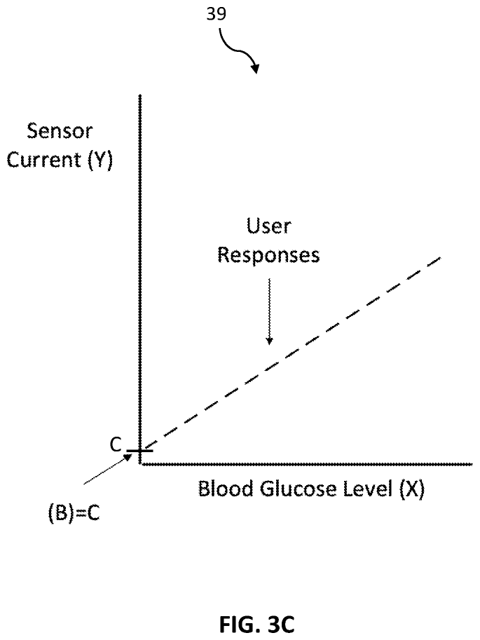

Referring now to FIG. 3A, a cross-section of a glucose-specific sensor 30 is illustrated, in accordance with some embodiments. The glucose-specific sensor 30 has a working electrode 31 which cooperates with a reference electrode 32 to provide an electrochemical reaction that can be used to determine glucose levels in a patient's blood or ISF. Although sensor 30 is illustrated with one working electrode 31 and one reference electrode 32, it will be understood that other embodiments may use multiple working electrodes, multiple reference electrodes, and counter electrodes. It will also be understood that sensor 30 may have different physical relationships between the working electrode 31 and the reference electrode 32. For example, the working electrode 31 and the reference electrode 32 may be arranged in layers, spiraled, arranged concentrically, or side-by-side. It will be understood that many other physical arrangements may be consistent with the disclosures herein.

The working electrode 31 has a conductive portion, which is illustrated for glucose-specific sensor 30 as conductive wire 33. This conductive wire 33 can be for example, solid platinum, a platinum coating on a less expensive metal, carbon, or plastic. In other words, conductive wire 33 may be a conductive surface (i.e., conducting layer) of a wire in some embodiments. It will be understood that other electron conductors may be used consistent with this disclosure. The working electrode 31 also has an interference layer 34, an enzyme layer 35, and a glucose limiting layer 36. Glucose limiting layer 36 may be used to limit contaminations and the amount of glucose that is received into the enzyme membrane 35. In this disclosure, the glucose limiting layer may also be referred to as a glucose limiting membrane, the enzyme layer may also be referred to as an enzyme membrane, and the interference layer may also be referred to as an interference membrane.

The glucose specific sensor 30 is inserted in a patient to contact a patient's body fluid, such as blood or ISF. The outer glucose limiting layer 36 is constructed to pass a determined level of glucose from the blood or ISF to the enzyme layer, but effectively blocks most, such as over 95%, of the active electrochemical contaminants in the blood or ISF from the enzyme layer 35. The enzyme layer 35 includes the GOx enzyme, which reacts with the glucose to generate H₂O₂. The interference layer 34, which is between the enzyme layer 35 and the conductive surface of the substrate (conductive wire 33), blocks nearly all of the active electrochemical in the enzyme layer 35 from ever reaching the conductive surface, while freely passing the H₂O₂ to the conductive surface of the conductive wire 33. In some cases, the interference layer 34 provides the sensor 30 with an electrical sensitivity of over 1000 nA/mM.

The H₂O₂ reacts with the conductive surface, which is typically platinum, with the reaction generating free electrons that flow on the conductive surface of the conductive wire 33 to the sensor's electronics. The current generated from the H₂O₂ reaction is proportional to the concentration of glucose in the patient's blood or ISF and is referred to as the “glucose current.” Although the glucose limiting layer 36 has blocked most (e.g., over 95%) of the active electrochemical contaminants, and most the rest of the contaminants were blocked by the interference layer 34, it is possible that an insignificant number of active electrochemical contaminants pass to the conductive surface (conductive wire 33), where they react with the conductive surface to create a current referred to as the “contaminant current.” However, due to the effective blocking of the active electrochemical contaminants by the glucose limiting layer 36 and the interference layer 34, the contaminant current is so small that it has no meaningful effect in calculating and communicating a blood glucose level. Depending on the specific construction of the glucose-specific sensor 30, the glucose current can be 100, 500, 1000 or even 10,000 times larger than the contaminant current. It is this incredibly high signal (glucose current) to noise (contaminant current) ratio that enables the total current flowing from the sensor 30 to attributed solely to the glucose current. In some embodiments, the glucose limiting layer 36 (GLL) blocks greater than 95% of the active electrochemical contaminants from entering the enzyme layer 35, thereby passing less than 5% of the active electrochemical contaminants into the enzyme layer. In some embodiments, the GLL blocks greater than 97% of acetaminophen from the patient from entering the enzyme layer, thereby passing less than 3% of the patient's acetaminophen into the enzyme layer. In some embodiments, the GLL blocks greater than 99% of ascorbic acid from the patient from entering the enzyme layer, thereby passing less than 1% of the patient's ascorbic acid into the enzyme layer.

In some embodiments, the glucose limiting layer 36 is formulated and constructed with polyurethane having a molecular weight greater than 100,000 Daltons that is physically crosslinked with a water-soluble polymer having a molecular weight greater than 100,000 Daltons. The polyurethane may be, for example, a thermoplastic silicone polyether polyurethane or a thermoplastic silicone polycarbonate polyurethane. In some embodiments, the water-soluble polymer of the glucose limiting layer may comprise polyacrylic acid, polyvinyl alcohol (PVA), polyvinylpyrrolidone (PVP) or poly(ethylene oxide) (PEO) or other water-soluble polymers to physically crosslink with the polyurethane. This construction enables the glucose limiting layer to be highly effective at blocking or rejecting active electrochemical contaminants, such as acetaminophen, uric acid, and ascorbic acid. The blocking or rejecting may be due to bonding of the contaminants or due to charge-based interactions. For example, contaminants may become hydrogen bonded to PVP, thus being prevented from passing through the glucose limiting layer 36. In another example, PVA or polyacrylic acid may serve as charge repulsion materials, inhibiting certain contaminants from passing through.

In experimental testing performed in relation to this disclosure, it was observed and tested that the glucose limiting layer 36 can, by itself, reject or block over 95% of the active electrochemical contaminants in the patient's blood. Indeed, as shown in Table 1 below, the glucose limiting layer 36 blocked about 97% of acetaminophen and 99.5% of the ascorbic acid compared to a control scenario of a bare sensor without the glucose limiting layer. Similar blocking rates and effectiveness were observed for nearly all active electrochemical contaminants.

TABLE 1

| | Acetaminophen | Ascorbic Acid |
|---|---|---|
| | sensitivity | Sensitivity |
| Sensor Profiles | (nA/mM) | (nA/μM) |
| Bare Sensors (n = 12) | 916 ± 157 | 873.6 ± 146 |
| Sensors with Glucose Limiting Layer | 20.07 ± 2.06 | 4.76 ± 0.51 |
| (n = 12; GLL of polyurethane of | (blocked 97%) | (blocked 99.5%) |
| MW = 200,000 Daltons crosslinked | | |
| with PVP of MW = 1,300,000 | | |
| Daltons) | | |

Further, the glucose limiting layer 36 may substantially limit or set the amount of glucose that can reach the enzyme membrane 35, for example only allowing about 1 of 1000 glucose molecules to pass. By strictly limiting the amount of glucose that can reach the enzyme membrane 35, linearity of the overall response is improved. The glucose limiting layer 36 also permits oxygen to travel to the enzyme membrane 35. The key chemical processes for glucose detection occur within the enzyme membrane 35. Typically, the enzyme membrane 35 has one or more glucose oxidase enzymes (GOx) dispersed within the enzyme membrane 35. When a molecule of glucose and a molecule of oxygen (O₂) are combined in the presence of the glucose oxidase, a molecule of gluconate and a molecule of hydrogen peroxide are formed. The hydrogen peroxide then generally disperses both within the enzyme membrane 35 and into interference membrane 34 (which may also be referred to in this disclosure as an interference layer).

Three performance characteristics are important to the effectiveness and desirability of the interference layer 34: its sensitivity, stability, and contaminant blocking. Sensitivity is a measure of the level of hydrogen peroxide that must be received at the working electrode surface passing through the interference membrane 34 to generate sufficient free electrons for an accurate measurement. Generally, it is highly desirable for the interference layer 34 to have greater sensitivity, as this allows for operation at lower voltages and bias currents and reduces the level of noise in the detection signal, which leads to a more accurate measurement. In embodiments, interference layer 34 is made sufficiently thin to pass sufficient H₂O₂ to generate at least 1000 nA/mM of H₂O₂, such as 1000 to 3000 nA/mM. With such incredibly high sensitivity, the signal generated responsive to the H₂O₂ overwhelms any noise generated from the active electrochemical contaminants. Said another way, any electrical signal generated due to the active electrochemical contaminants is de minimus and has no practical effect on the glucose reading presented by the CGM.

Better stability makes for a more desirable interference layer 34. Stability refers to how the hydrogen peroxide reaction changes over time. More stability results in less complicated calibration as well as a sensor that has a longer useful life with more reliable results. Accordingly, it is desirable for the interference layer 34 to have better sensitivity and stability characteristics.

In some embodiments, the interference membrane 34 is nonconductive of electrons, but is conductive of ions. In practice, a particularly effective interference membrane may be constructed using, for example, poly-ortho-aminophenol (POAP, or poly(o-aminophenol)), polypyrrole, polyaniline, and/or poly(phenylenediamine). For example, a polymer made of monomers selected from aminophenols, aniline, phenylenediamine, pyrrole or combinations thereof may be used in interference membrane 34. In a specific example, the interference membrane may include pyrrole and phenylenediamine. The monomer(s) may be deposited onto the conductive wire 33 (e.g., platinum or platinum-coated) using an electrodeposition process, at a thickness that can be precisely controlled to enable a predictable level of hydrogen peroxide to pass through the interference membrane 34 to the conductive wire 33. Further, the pH level and/or a salt concentration of the monomer solution may be adjusted to set a desirable permselectivity for the interference membrane 34. For example, the pH and/or salt concentration may be advantageously adjusted to significantly block the passage of larger molecules such as acetaminophen, thereby reducing contaminants that can reach the conductive wire 33. It will be understood that other materials may be used. For example, the interference layer may include a polymer that has been electropolymerized from: aniline, naphthol, phenylenediamine, 2-aminophenol, 3-aminophenol, 4-aminophenol, m-phenylenediamine, o-phenylenediamine, p-phenylenediamine, pyrrole, derivatized pyrrole, aminophenylboronic acid, thiophene, porphyrin, aniline, phenol, or thiophenol or blends thereof.

Advantageously, by adjusting the pH and/or salt concentration of the monomer solution for forming the interference layer, the permselectivity of the layer can be adjusted to further block the few contaminants that passed into the enzyme layer 35, and the level of H₂O₂ that passes through to the conductive surface can be increased by making the interference layer 34 thinner. In this way, the level of electrical signal attributable to the active electrochemical contaminants can be ignored, and the electric current on the conductor can be considered fully due to the presence of glucose. The sensor 30 is therefore a glucose-specific sensor and is not subject to the variations caused by varying amounts of active electrochemical contaminants in a user's body. This advancement enables the elimination of local finger-prick calibration, and instead allows the sensor 30 to be calibrated in-factory only.

The interference membrane 34 is layered between the electrical conducting wire 33 (e.g., platinum wire) and the enzyme membrane 35 in working electrode 31. Generally, the interference membrane 34 is applied as a monomer, with selected additives, and then polymerized in situ on the conductive wire 33.

This interference membrane 34 may be electrodeposited onto the electrical conducting wire 33 in a very consistent and conformal way, thus reducing manufacturing costs as well as providing a more controllable and repeatable layer formation. The interference membrane 34 is nonconducting of electrons, but will pass ions and hydrogen peroxide at a preselected rate. Further, the interference membrane 34 may be formulated to be permselective for particular molecules. In one example, the interference membrane 34 is formulated and deposited in a way to restrict the passage of active molecules, which may act as contaminants to degrade the electrical conducting wire 33, or that may interfere with the electrical detection and transmission processes.

Advantageously, the interference membrane 34 provides reduced manufacturing costs as compared to known insulation layers and is enabled to more precisely regulate the passage of hydrogen peroxide molecules to a wide surface area of the underlying conductive wire 33. Further, formulation of the interference membrane 34 may be customized to allow for restricting or denying the passage of certain molecules to underlying layers, for example, restricting or denying the passage of large molecules or of particular target molecules.

Interference membrane 34 is a coating fully surrounding the platinum wire (i.e., conductive wire 33). In this way, the expense and uncertainty of providing a window through an insulating layer as in conventional sensors is avoided. Accordingly, the interference membrane 34 may be precisely coated or deposited over the conductive wire 33 in a way that has a predictable and consistent passage of hydrogen peroxide. Further, the allowable area of interaction between the hydrogen peroxide and the surface of the conductive wire 33 is dramatically increased, as the interaction may occur anyplace along the conductive wire 33. The interference membrane 34 enables an increased level of interaction between the hydrogen peroxide molecules in the surface of the conductive wire 33 such that the production of electrons is substantially amplified over prior art working electrodes. The interference membrane enables the sensor to operate at a higher electron current, reducing the senor's susceptibility to noise and interference from contaminants, and further enabling the use of less sophisticated and less precise electronics in the housing. In one non-limiting example, the ability to operate at a higher electron flow allows the sensor's electronics to use more standard operational amplifiers (op-amps), rather than the expensive precision op-amps required for prior art sensor systems. The resulting improved signal to noise ratio allows enable simplified filtering as well as streamlined calibration.

Further, during the manufacturing process it is possible to remove oxidation on the outer surfaces of the conductive wire 33 prior to depositing the interference membrane 34. Since the interference membrane 34 acts to seal the conductive wire 33, the level of oxidation can be dramatically reduced, again allowing for a larger interaction surface and further amplification of the glucose signal, resulting in higher electron flow and enabling a higher signal to noise ratio. In this way, the interference layer of the present disclosure prevents fouling of the platinum's electrical interface by eliminating undesirable oxidative effects.

Sensor 30 also has a reference electrode 32 separate from working electrode 31. In this way, the manufacture of the working electrode is simplified and can be performed with a consistency that contributes to dramatically improved stability and performance. In some embodiments, the reference electrode 32 is constructed of silver or silver chloride 37.

Sensor 30 enables an accurate and stable blood glucose reading without local user calibration. That is, due to the high level of contaminant rejection, the impact from noise and contaminant generated currents are eliminated, or at least nearly eliminated. It is the combination of the glucose limiting layer 36, enzyme layer 35, and interference layer 34 that cooperate and aggregate to remove the need for local user calibration, such as finger prick calibration. The extremely low (or close to zero) in-vivo baseline is achieved with the novel sensor membrane structures and processes described herein. The in-vivo interference compounds are blocked by the combination of all three membrane layers and the suitable amount of glucose is permeable into the sensor, which results a highly accurate and stable in-vivo glucose specific sensor. For example, the glucose limiting layer cooperates with the interference layer to block over 99%, or over 99.9%, or over 99.99% of active electrochemical contaminants from passing to the working wire's conductive surface. As described herein, active contaminants are typically present that produce an electric current that interferes with the electric signal generated due to the presence of glucose in the user's body fluid, such as ISF or blood. However, the glucose-specific sensor 30 is constructed to eliminate, or nearly eliminate, the active contaminants, and therefore eliminate, or nearly eliminate, any noise or negative electrochemical influence from active contaminants. Indeed, the use of the glucose-specific sensor 30 during testing related to the present disclosure has been found to reduce the impact of electro-active contaminants by up to 500 to 1000 times the amount of contaminants compared to known sensors. Accordingly, the aggregate electric current noise from all contaminants is less than about 0.5% of the electric current generated due to the presence of glucose in the user's blood, and in many cases is less than 0.1%. For example, greater than 99% of the electric current may be generated responsive to the in-vivo patient glucose concentration due to blocking of active electrochemical contaminants, such that less than 1% or less than 0.5% or less than 0.1% of the generated electric current is due to electrochemical reactions of the active electrochemical contaminants.

The sensor's zero-baseline structures enable a factory calibration using the glucose-specific monitoring product without additional SMBG finger prick calibration. With a near zero baseline, it is possible to more accurately calculate the glucose sensor without any SMBG in-vivo calibration. Further, sensors are often bulk-packed for distribution, for example, in sets of 25, 100 or even 1000. As a result of the near-zero intercept of the present sensors, any of the sensors in a distribution set of glucose-specific sensors may be used in any patient without any local finger-prick calibration.

Referring now to FIG. 3B, a sensitivity chart 38 is illustrated. Sensitivity chart 38 is similar to sensitivity chart 18 discussed with reference to FIG. 1B but shows the zero baseline results of the glucose specific sensor, such as glucose-specific sensor 30. In particular, the user response dotted line can represent many different users, or the same user at many different times. Either way, the user response is nearly the same in all cases, and the user response crosses the X and Y axis at zero, which is referred to as the “intercept.” Accordingly, the glucose specific sensor 30 has a zero or near-zero intercept, and therefore does not need local user calibration, but can rely entirely upon factory calibration prior to shipment to the user. The generated electric current in response to an in-vivo glucose concentration of the patient may be, for example, less than 0.2 nA when the actual in-vivo glucose concentration in the patient is zero. Further, due to the consistent user response of the glucose specific sensor, trustworthiness and accuracy in the resulting glucose reading is increased.

Twenty-three glucose specific sensors were made, tested and factory calibrated as discussed with reference to FIGS. 3A and 3B. The in-vivo glucose sensitivity and in-vivo baseline were calculated for the 23 glucose specific sensors and inserted in humans interstitially as shown in Table 2 below. Sensitivity for the sensors was established using a best fit calculated using reference SMBG (finger prick) points. All 23 of the glucose specific sensors had exceptional glucose sensitivity of between about 0.03 and 0.05 nA/mg/dL. Further, the glucose specific sensors had an average accuracy of nearly 93%. The test glucose specific sensors had a test software algorithm that enabled the sensor data to be evaluated at several baseline correction values in the range of −3 nA to +5 nA. Consistently the best fitting sensitivity was found when the baseline correction amperage was set to 0.0. Accordingly, testing of over 23 glucose specific sensors firmly established that the sensors had exceptionally accurate sensitivity with no baseline compensation. Because the baseline value was essentially zero for all the sensors, these sensors do not need to be calibrated to an individual user, but can be used by anyone after only a simple factory calibration.

To obtain the best in-vivo accuracy for each sensor against the reference SMBG values, the in-vivo baselines for each sensor was determined. Most of the 23 sensors had the in-vivo baseline value to be zero, the remaining sensors also had the in-vivo baseline very close to zero. Those close to zero baseline values demonstrated that the glucose specific sensors had true specific response towards glucose and glucose only.

TABLE 2

| | In-Vivo | | |
|---|---|---|---|
| | Glucose | In-Vivo | In-Vivo |
| | Sensitivity | Baseline | Accuracy |
| Sensor | (nA/mg/dL) | (nA) | (MARD) |
| Sensor01 | 0.039 | 0 | 5.98% |
| Sensor02 | 0.033 | 0 | 7.28% |
| Sensor03 | 0.05 | −0.1 | 9.18% |
| Sensor04 | 0.048 | 0 | 5.49% |
| Sensor05 | 0.044 | 0 | 7.81% |
| Sensor06 | 0.046 | 0 | 8.17% |
| Sensor07 | 0.04 | 0 | 5.53% |
| Sensor08 | 0.039 | 0 | 8.58% |
| Sensor09 | 0.036 | 0.1 | 6.92% |
| Sensor10 | 0.041 | 0 | 6.28% |
| Sensor11 | 0.037 | −0.1 | 6.87% |
| Sensor12 | 0.043 | 0 | 7.61% |
| Sensor13 | 0.042 | 0 | 5.80% |
| Sensor14 | 0.042 | 0 | 8.39% |
| Sensor15 | 0.043 | 0 | 9.10% |
| Sensor16 | 0.038 | 0 | 8.32% |
| Sensor17 | 0.042 | −0.1 | 7.20% |
| Sensor18 | 0.04 | 0 | 8.15% |
| Sensor19 | 0.037 | −0.1 | 8.61% |
| Sensor20 | 0.041 | −0.1 | 9.19% |
| Sensor21 | 0.039 | 0 | 7.72% |
| Sensor22 | 0.054 | 0 | 9.70% |
| Sensor23 | 0.045 | −0.1 | 9.35% |
| AVERAGE | | −0.02 | 7.70% |

(MARD = Mean absolute relative difference)

Referring now to FIG. 3C, a sensitivity chart 39 is illustrated. Sensitivity chart 39 is similar to sensitivity chart 38 discussed with reference to FIG. 3B but shows the baseline results of the glucose specific sensor, such as glucose-specific sensor 30. In particular, the user response dotted line can represent many different users, or the same user at many different times. Either way, the user response is nearly the same in all cases, and the user response crosses the Y axis at a baseline value C, which is the intercept. C is a constant. Accordingly, the glucose specific sensor 30 does not need local user calibration, but can rely entirely upon factory calibration prior to shipment to the user to remove most if not all of the value C. Further, due to the consistent user response of the glucose specific sensor, trustworthiness and accuracy in the resulting glucose reading is increased. In this way, the glucose-specific sensor provides that all users, or any one user at all times, will have a user response having a constant baseline. It is this constant baseline that enables the avoidance of local user calibration. It will be understood that in some cases the baseline may be zero, near zero, or at a constant current on the Y-axis.

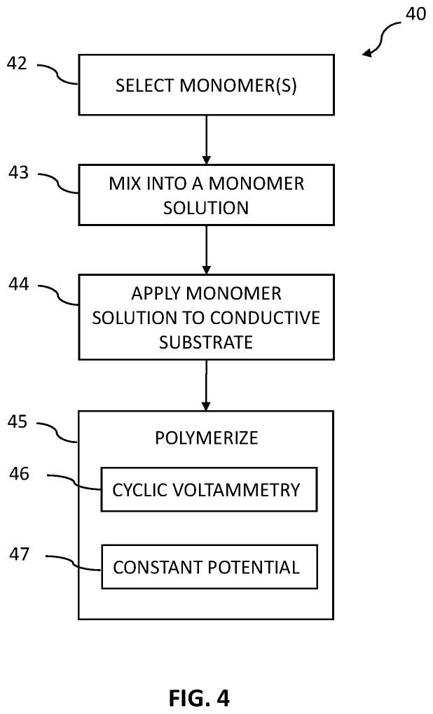

Referring now to FIG. 4, a flowchart of a process 40 for making an interference layer for a working wire is described. In some embodiments of the interference layer, an interference compound is electrodeposited onto a conductive substrate, and the enzyme layer is applied over the interference compound. The interference compound is nonconducting, ion passing, and permselective according to a particular molecular weight. Further, it is electrodeposited in a thin and conformal way, enabling more precise control over the flow of hydrogen peroxide from the enzyme layer to the conductive substrate. In some embodiments, the interference material is made by mixing a monomer with a mildly basic buffer, and then electropolymerizing the mixture into a polymer. The buffer may include a salt, such as NaCl or KCl, to adjust the pH of the monomer solution and consequently tune the electropolymerization process to adjust the permselectivity of the interference layer.

In some embodiments, the interference membrane is nonconductive of electrons, but is conductive of ions. An interference membrane may be constructed using monomers that include one or more of, for example: pyrrole, phenylenediamines (PDA), aminophenols, or aniline. The monomers are polymerized on the conductive substrate. For example, pyrrole may be polymerized to form polypyrrole, PDA may be polymerized to form poly(phenylenediamine), or ortho-aminophenol (o-aminophenol) may be polymerized to form Poly-Ortho-Aminophenol (POAP), or aniline may be polymerized to form polyaniline. Using phenylenediamine as an example, p-phenylenediamine may be deposited onto the conductive wire 33 (e.g., platinum or platinum-coated) using an electrodeposition process, at a thickness that can be precisely controlled to enable a predictable level of hydrogen peroxide to pass through the interference membrane 34 to the conductive wire 33. Further, the pH level and/or use of salts in the monomer solution containing the p-PDA may be adjusted to set a desirable permselectivity for the interference membrane 34. For example, the pH and/or use of salts (choice of salts and concentration in the monomer solution) may be advantageously adjusted to significantly block the passage of larger molecules such as acetaminophen, thereby reducing contaminants that can reach the conductive wire 33. It will be understood that other materials may be used. For example, the interference layer may include a polymer that has been electropolymerized from: aniline, naphthol, phenylenediamine, 2-aminophenol, 3-aminophenol, 4-aminophenol, m-phenylenediamine, o-phenylenediamine, p-phenylenediamine, pyrrole, derivatized pyrrole, aminophenylboronic acid, thiophene, porphyrin, phenol, or thiophenol or blends thereof. It will be appreciated that other monomers may be used. In a more specific example, the monomer is 2-aminophenol and the buffer is phosphate buffered saline (PBS) at about 8 pH. The monomer and the buffer are mixed and electropolymerized into the polymer Poly-Ortho-Aminophenol (POAP). The POAP is then electrodeposited onto the conductive substrate. The permselectivity of the POAP may be adjusted by the pH of the buffer, for example by adding sodium hydroxide (NaOH) or hydrochloric acid (HCl).

Process 40 illustrates one example construction for the interference layer 34 where the interference membrane shall be described using phenylenediamines (PDAs) as an example. PDAs are non-conducting monomers and can be polymerized, such as using a solution or a mixture of solutions to facilitate polymerization. As illustrated in block 42, monomers are selected, such as PDAs, pyrroles, anilines, aminophenols or blends of these. A blend may include a main monomer with one or more co-monomers. The percentage of monomer to co-monomer may be, for example 80% main monomer to 20% co-monomer. In other embodiments, the main monomer can range from 20% to 80% compared to the amount of co-monomer. In a more specific example, the polymer of the interference layer is formed from a monomer and a co-monomer, the monomer being phenylenediamine and the co-monomer being pyrrole. In another example, the monomer is phenylenediamine and the co-monomer may include one or more of 2-aminophenol, 3-aminophenol, 4-aminophenol, m-phenylenediamine, o-phenylenediamine, p-phenylenediamine, pyrrole, derivatized pyrrole, or aniline. Block 42 may also involve selecting any additives to be used in the monomer solution. In block 43, the monomers are mixed into a monomer solution, such as with water, NaOH, HCl, or other solvents. In one example, the monomer concentration is prepared in the range of 1 to 200 mM. In some embodiments, a liquefying buffer solution is selected as a solvent for the purpose of both facilitating polymerization, and for enabling the PDAs to be mixed into a usable gel. Appropriate buffer solutions can be, for example, phosphate buffered saline (PBS) in the range of 10 to 200 mM. In one embodiment, the PDAs, buffer solution, and any other additives are mixed into a gel or paste for use in, for example, automated application processes.

This monomer solution gel or paste is then applied to the conductive substrate as illustrated in block 44 in a layer sufficiently thin to allow for a high level of passage of H₂O₂ as described earlier. Generally, this conductive substrate has a platinum outer surface onto which the gel is applied, for example by submerging, dipping, coating, or spraying. It will be appreciated that other processes can be used, such as electrodepositing or other deposition process. It is understood that the interference layer can be deposited in block 44 at a controlled temperature such as in the range of 20 to 60° C. depending on the methods and application process, and at pressures such as ambient pressure. Once the gel has been uniformly applied to the conductive substrate at a desired thickness, the monomers are polymerized to form PDA polymers as illustrated in block 45.

In some embodiments, the polymerization process of block 45 involves electropolymerization, which may involve a cyclic voltammetry process 46 or application of a constant potential 47, or both in combination. When used in combination, the cyclic voltammetry process 46 can be performed before or after the application of a constant potential 47. The cyclic voltammetry process 46 involves a window range, a start voltage, and a number of cycles. Each cycle, which is also referred to as a scan, involves increasing the voltage from zero to a particular positive voltage, then decreasing the voltage to a particular negative voltage, then returning the voltage to zero. In one example, the number of voltage cycles for which cyclic voltammetry is applied is increased compared to conventional voltammetry cycle numbers (e.g., 2 to 10 scans conventionally), and in some cases additional cycles added. Thus, in some embodiments cyclic voltammetry is applied for longer time and/or more periods than conventional methods. It has been found in the present disclosure that increasing the number of cycles to over 10 cycles results in an interference layer that enables protection against negative effects due to exposure to a sterilizing gas. In some embodiments, a scan rate of the cyclic voltage application in the range of 2 to 200 mV/s, a starting voltage in the range of −0.5 to 0.5V as well as a voltage range of −1 to 2 V vs. Ag/AgCl electrode may be used, but it will be understood that these window ranges may be adjusted to the particular formulations and application-specific requirements. Furthermore, a constant potential polymerization process 47 may be used instead of, or along with, the cyclic voltammetry process 46. In some embodiments, a constant voltage in the range of +100 to 600 mV vs. Ag/AgCl electrode, applied for a period in the range of 100-2000 seconds, results in an interference layer that has been found to enable desirable contaminant protection as well as advantageous control of the passage of hydrogen peroxide. The application of constant potential can beneficially stabilize the interference layer, improving performance and reducing the need for in-vivo calibrations.

The interference layer beneficially serves as a microporous material, where “pores” in polymer strands of the layer allow certain sizes of molecules to pass through. By controlling the sizes and amount of the “pores,” and the thickness of the interference layer, the sizes of contaminants that will be blocked by the interference layer can be controlled while still enabling H₂O₂ to pass through. This permselectivity can be designed into the interference layer through the electropolymerization process that is used to form the interference layer. The interference layer is beneficially formed in situ on the conductive substrate, enabling the interference layer to conform to the conductive substrate.

In some embodiments, salts (e.g., NaCl or KCl) can be utilized in the monomer solution to achieve a desired permeability of the interference layer as well as to improve the efficiency of the electropolymerization process. Regarding permeability, the type of salt can be chosen to achieve desired sizes of the “pores” of the layer, and the concentration of salts can be tuned to achieve a desired amount of “porosity.” For example, decreasing the concentration of salts will make the interference layer less permeable to contaminants. Embodiments balance the concentration of salts in the monomer solution to achieve sufficient blocking of contaminants while maintaining permeability to H₂O₂. The concentration of salts in the monomer solution can also be adjusted to affect the efficiency of the electropolymerization process and consequently the uniformity of the layer. In particular, the salts change the electrical conductivity and osmolality of the solution (which may have deionized water or PBS as a primary solvent), where a higher conductivity will increase the electrical current that flows through the solution during the electropolymerization process. When voltage is applied during electropolymerization, such as through cycle voltammetry 46 and/or constant potential 47 of FIG. 4, electrical current flows through the monomer solution and causes monomers to polymerize. During the polymerization, the polymerized material on the conductive substrate builds up, where the layer will build up irregularly over the surface of the conductive substrate. Areas of less material build-up can create a selectively permeable network for molecules (e.g., H₂O₂) to pass through or to be blocked (e.g., contaminants larger than a particular size). The osmolality and electrical conductivity of the monomer solution impact the electrical current flow and therefore the polymerization rate, consequently affecting the permeability of the interference layer.

In some embodiments, the electropolymerization parameters (e.g., rate of voltage changes and voltage window) can be adjusted to achieve a desired thinness of the interference layer to preserve hydrogen peroxide permeability. The electropolymerization process is self-limiting in that as the layer builds up, the layer becomes an insulating layer which causes the current flow to decrease and thus the polymerization to decrease. Embodiments advantageously enable thinner layers to be achieved than conventional self-limiting electropolymerization processes, by adjusting electrical properties of the monomer solution. Methods may include adjusting a salt concentration of the phosphate buffered saline to adjust an electrical conductivity of the solvent for the electropolymerizing. For example, increasing the salt concentration can increase the osmolality and consequently the electrical conductivity. Higher electrical conductivity can make the electropolymerization more efficient, such as by achieving a polymerization rate that self-limits at a target thickness (e.g., 0.1 μm to 2.0 μm). This thickness can be designed to be thin enough to enable H₂O₂ to travel through the interference layer while contaminants are blocked.

In some embodiments, holding a constant potential for a designated period of time during the electropolymerization—such as approximately 30 seconds, or 30 seconds to two minutes, or at least 100 seconds, or at least two minutes, or ten to thirty minutes—is uniquely used in the present methods to stabilize the interference layer. The application of constant potential beneficially stabilizes the interference layer by allowing reactions of any unreacted material to be completed and/or allowing unneeded material to leave the layer. The stabilization can be controlled by the voltage level and length of time of the constant potential.

Table 3 shows example experimental results for working wires having an interference layer as disclosed herein. Test group A had an interference layer made of PDA and pyrrole, and test group B had an interference layer made of PDA. As can be seen by Table 3, the presence of the interference layer in both test groups improved the glucose sensitivity and greatly blocked acetaminophen compared to the control samples.

TABLE 3

| Sample Data | Glucose Sensitivity | Acetaminophen (Interference) |
|---|---|---|
| (n = 10) | (nA/mg/dL) | Sensitivity (nA/mM) |
| Control: Without | 0.049 +/− 0.003 | 25.87 +/− 1.69 |
| Interference Layer | | |
| Group A: With | 0.058 +/− 0.006 | 0.26 +/− 0.21 |
| Interference Layer | | |
| (PDA/Pyrrole) | | |
| Group B: With | 0.055 +/− 0.012 | 1.42 +/− 1.71 |
| Interference Layer | | |
| (PDA) | | |

In some embodiments, the stability of the interference layer is controlled by the monomer concentrations prior to electropolymerization. In some embodiments, the stability of the interference layer is controlled by the electropolymerization temperature. In some embodiments, the stability of the interference layer is controlled by the additives of the electropolymerization. The additives may include, for example, phosphate buffered saline, sodium chloride (NaCl), or potassium chloride (KCl).

It will be understood that other processes may be used to polymerize the monomers to form the PDA polymers. Once the interference layer has been fully polymerized, then the enzyme layer may be layered or deposited over the interference layer. A working wire may then be completed by adding additional layers, such as a glucose limiting layer or protective layer.

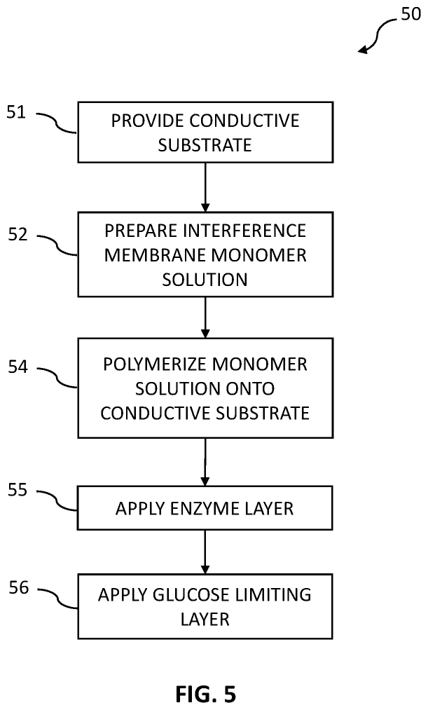

Referring now to FIG. 5, a flowchart of a process 50 for manufacturing a working wire is provided. In process 50, a conductive substrate is selected and provided in block 51. This conductive substrate may be solid platinum, or may be a less expensive substrate coated with a layer of platinum. In some embodiments, the substrate may be, for example tantalum, a Co—Cr alloy, or plastic. It will be appreciated that other substrates may be used. In some cases, a carbon conductive substrate may be provided. As shown in block 52, the interference membrane is prepared as described in FIG. 4 and throughout this disclosure and may include in some cases a buffer solution having a salt. In some embodiments, the interference membrane compound will be produced as a gel or paste that may be applied to the substrate during an automated manufacturing process. The interference membrane compound is then applied to the conductive surface as illustrated in block 54. The interference membrane compound may be applied by, for example, dipping, coating, a deposition process (e.g., electropolymerization), or spraying. It will be appreciated that other application processes may be used. The interference membrane compound, which is composed of monomers, is then polymerized, for example using cyclic voltammetry with longer times or periods than conventional cyclic voltammetry, and/or by a constant potential as described with reference to FIG. 4.

After the interference layer has been polymerized, an enzyme layer is applied as shown in block 55, such as an enzyme layer having glucose oxidase (GOx), such as GO₂. It will be appreciated that other enzymes may be used depending upon the particular substance to be monitored. In some cases, a glucose limiting layer can be applied over the enzyme layer as shown in block 56. This glucose limiting layer may not only be used to limit the level of glucose passing into the enzyme layer, but it can add a layer of protection, and some biocompatibility to the overall working wire.

In embodiments, a glucose-specific sensor for in-vivo use in a patient has a glucose limiting layer, an enzyme layer, an interference layer, and a substrate. The glucose limiting layer comprises a polyurethane with a molecular weight greater than 100,000 Daltons that is physically crosslinked with a water-soluble polymer having a molecular weight greater than 100,000. The enzyme layer comprises glucose oxidase (GOx) for reacting with in-vivo glucose in body fluid from the patient to generate hydrogen peroxide (H₂O₂). The body fluid may be ISF, for example. The interference layer comprises a polymer formed from pyrrole, phenylenediamine (PDA), aminophenol, aniline, or combinations thereof, wherein the enzyme layer is between the interference layer and the glucose limiting layer. The substrate has a conductive surface adjacent the interference layer for carrying an electric current generated in response to an in-vivo glucose concentration of the patient.

In some embodiments, the water-soluble polymer of the glucose limiting layer comprises polyacrylic acid, polyvinyl alcohol, polyvinylpyrrolidone, poly(ethylene oxide), or combinations thereof to physically crosslink with the polyurethane. In some embodiments, the polyurethane of the glucose limiting layer is a thermoplastic silicone polyether polyurethane or a thermoplastic silicone polycarbonate polyurethane. In some embodiments, the water-soluble polymer may be polyvinylpyrrolidone that is cross-linked with a thermoplastic silicone polyether polyurethane or a thermoplastic silicone polycarbonate polyurethane. In some embodiments, the polymer of the interference layer is electropolymerized on the substrate. In some embodiments, the polymer of the interference layer is formed from a monomer and a co-monomer, the monomer being p-phenylenediamine; where the co-monomer comprises 2-aminophenol, 3-aminophenol, 4-aminophenol, m-phenylenediamine, o-phenylenediamine, pyrrole, derivatized pyrrole, or the aniline.

In some embodiments, the body fluid in the patient further comprises active electrochemical contaminants, the glucose limiting layer blocks greater than 95% of the active electrochemical contaminants from entering the enzyme layer, and the interference layer substantially blocks the active electrochemical contaminants that have entered the enzyme layer from passing to the conductive surface. In some embodiments, less than 1% of the generated electric current is due to electrochemical reactions of the active electrochemical contaminants. In some embodiments, the generated electric current is less than 0.2 nA when the in-vivo glucose concentration is zero.

In embodiments, a glucose-specific sensor for in-vivo use in a patient has a glucose limiting layer, an enzyme layer, an interference layer, and a substrate. The glucose limiting layer comprises a polyurethane with a molecular weight greater than 100,000 Daltons that is physically crosslinked with a water-soluble polymer. The enzyme layer comprises glucose oxidase (GOx) for reacting with in-vivo glucose in body fluid from the patient to generate hydrogen peroxide (H₂O₂). The body fluid may be ISF, for example. The interference layer comprises pyrrole and phenylenediamine (PDA), wherein the enzyme layer is between the interference layer and the glucose limiting layer. The substrate has a conductive surface adjacent the interference layer for carrying an electric current in response to an in-vivo glucose concentration of the patient.

In some embodiments, the water-soluble polymer has a molecular weight greater than 100,000 Daltons. In some embodiments, the polyurethane of the glucose limiting layer is a thermoplastic silicone polyether polyurethane or a thermoplastic silicone polycarbonate polyurethane. In some embodiments, the interference layer further comprises a co-monomer polymerized with the pyrrole and the PDA, the co-monomer being 2-aminophenol, 3-aminophenol, 4-aminophenol, m-phenylenediamine, o-phenylenediamine, p-phenylenediamine, or aniline.

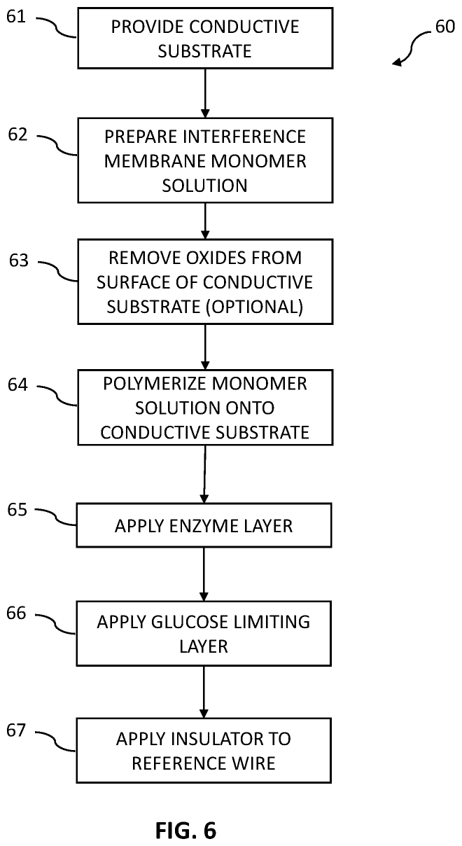

It will be appreciated that alternative compounds may be used to form the interference layer as described above. Referring now to FIG. 6, a flowchart of a process 60 for formulating and applying the interference membrane (i.e., interference layer) to a working wire of a continuous glucose monitor is illustrated. As shown in block 61, a conductive substrate is provided. This conductive substrate may be in the form of an elongated wire, but it will be appreciated that the conductive substrate can be provided in other forms, such as printed or in the form of conductive pads. In some embodiments, the conductive substrate is a solid platinum wire, a less expensive wire that has been coated with platinum, or as disclosed herein, the conductive substrate may be a conductive carbon compound coated on a plastic substrate. It will be appreciated that other conductive substrates may be used.

As shown in block 62, the interference membrane compound is prepared. This compound is formulated to be 1) non-electrically conducting; 2) ion passing; and 3) permselective. The interference layer may also provide protections against negative effects of EtO, and in some cases, exhibits improved stability and sensitivity after exposure to EtO gas. Further, the compound is particularly formulated to be electrodeposited in a thin and uniform layer, and has a thickness that is self-limiting due to the nature of electrically driven cross-linking. In this way, the compound may be applied in a way that provides a well-controlled regulation of hydrogen peroxide molecule passage using a simple and cost-effective manufacturing processes. Further, the passage of the hydrogen peroxide can occur over a much larger surface area as compared to prior art working wires.

Generally, the characteristics of the present interference membranes identified above can be formulated by mixing a monomer with a mildly basic buffer and converting the monomer into a more stable and usable polymer by applying an electropolymerization process. In one formulation:
a) Monomer: e.g., 2-aminophenol, 3-aminophenol, 4-aminophenol, aniline, Naphthol, m-phenylenediamine, o-phenylenediamine, p-phenylenediamine, pyrrole, derivatized pyrrole, aminophenylboronic acid, thiophene, porphyrin, phenol, or thiophenol or blends thereof
b) Buffer (solvent): e.g., Phosphate Buffered Saline (PBS) tuned to about 7 to about 10 pH, such as 7.5 to 9 pH, such as 8 pH by adding sodium hydroxide (NaOH). The buffer may also include a salt, such as NaCl or KCl, to adjust the electrical conductivity of the buffer.
c) Mix the monomer and buffer and apply to the conductive substrate.
d) Electropolymerize to create a polymer; e.g., poly(phenylenediamine), polypyrrole, polyaniline, and/or Poly-Ortho-Aminophenol (POAP).

In a particular embodiment of the formulation set out above, 2-aminophenol monomer is mixed with a PBS buffer being mildly basic at a pH 8. The pH of the PBS buffer is adjusted using an additive, such as sodium hydroxide. It will be understood that the pH may be adjusted to create alternative formulations consistent with this disclosure. For example, the pH of the compound may be adjusted such that the permselectivity of the resulting POAP (or other polymer(s) being formed, such as poly(PDA), polypyrrole, and/or polyaniline) can be modified. More particularly, the POAP may be formulated to have a defined molecular weight cutoff. That is, by adjusting the pH of the formulation, the POAP may be modified to substantially restrict the passage of molecules having a molecular weight larger than the cutoff molecular weight. Accordingly, the POAP can be modified according to the molecular weight of the contaminants that need to be restricted from reaching the platinum wire. It will also be understood that other monomers may be selected, and these alternative monomers may provide the desired functional characteristics at a different pH. The 2-aminophenol and PBS mixture is electropolymerized into POAP. It will be understood that other additives may be used such as NaCl, KCl, NaOH or HCl.

Optionally, the oxides or oxide layers may be removed from the surface of the conductive platinum substrate as illustrated in block 63. As described earlier in this disclosure, these oxides or layer of oxides dramatically restrict the surface area available to the hydrogen peroxide to react with the platinum. By removing these oxides or oxide layers, for example by chemical etching or physical buffing, a less contaminated conductive wire may be provided for coating. In this way, the surface area of the substrate (e.g., platinum) available for hydrogen peroxide interaction is dramatically increased, thereby increasing the overall electrical sensitivity of the sensor.

The interference compound is then applied to the conductive substrate and polymerized as shown in block 64. In one particular application, the interference compound is electrodeposited onto the conductive substrate, which deposits the compound in a thin and uniform layer. Further, the electrodeposition process facilitates a chemical cross-linking of the polymers as the monomer solution is deposited.

As described above, the interference membrane has a compound that is self-limiting in thickness. The overall allowable thickness for the membrane may be adjusted according to the ratio between the monomer and the buffer, as well as the particular electrical characteristics used for the electropolymerization process. In example embodiments, the thickness of the interference membrane may be 0.1 μm to 2.0 μm. Also, the interference membrane may be formulated for a particular permselective characteristic by adjusting the salt concentration. It will also be understood that a cyclic voltammetry (CV) process may be used to electrodeposit the interference membrane compound, such as polypyrrole, poly(PDA), POAP, polyaniline or combinations thereof. A CV process is generally defined by having (1) a scanning window that has a lower voltage limit and upper voltage limit, (2) a starting point and direction within that scanning window, (3) the scan rate for each cycle, and (4) the number of cycles completed. It will be understood by one skilled in the art that these four factors can provide many alternatives in the precise application of the interference membrane compound. In one example, the following ranges are effective for the CV process to apply POAP to achieve improved contamination and hydrogen peroxide performance. Generally, adjustments were made in the present embodiments, compared to conventional CV techniques, to lengthen cyclic time periods, or increase the number of exposure periods, to provide enhanced performance.
Scanning window: −1.0V to 2.0V
Starting point: −0.5V to 0.5V
Scan Rate: 2-200 mV/s
Cycles: 5-50

In another example, the following ranges are effective for the electropolymerization process to apply phenylenediamine to a substrate to form an interference layer. The phenylenediamine may be a monomer for the monomer solution, that is mixed with co-monomers such as one or more of 2-aminophenol, 3-aminophenol, 4-aminophenol, m-phenylenediamine, o-phenylenediamine, pyrrole, derivatized pyrrole, or aniline.
Scanning window: −1.0V to 2.0V
Starting point: −0.5V to 0.5V
Scan Rate: 2-200 mV/s
Cycles: 5-50
Constant Potential: 0.7V to 0.9V (e.g., 0.8V) for 30 seconds to 5 minutes

As illustrated in block 65, the enzyme layer is applied, which includes the glucose oxidase, and then a glucose limiting layer is applied as shown in block 66. This glucose limiting layer, as discussed above, is useful to limit the number of glucose molecules that are allowed to pass into the enzyme layer.

Finally, as illustrated in block 67, an insulator may be applied to the reference wire. In many cases, the reference wire will be a silver/silver oxide wire, and the insulator will be an ion limiting layer that is nonconductive of electrons.

In embodiments, methods for making a glucose-specific sensor for in-vivo use in a patient involve mixing a monomer with a solvent to form a monomer solution and applying the monomer solution to a substrate having a conductive surface. The method also involves electropolymerizing the monomer to form a polymer on the substrate, the polymer being an interference layer for the glucose-specific sensor. An enzyme layer is formed on the interference layer, and a glucose limiting layer is formed on the enzyme layer.

In some embodiments, the monomer comprises pyrrole, phenylenediamine (PDA), aminophenol, aniline, or combinations thereof. In some embodiments, the monomer is p-phenylenediamine, and the monomer solution may comprise a co-monomer, the co-monomer comprising 2-aminophenol, 3-aminophenol, 4-aminophenol, m-phenylenediamine, o-phenylenediamine, pyrrole, derivatized pyrrole, or aniline. In some embodiments, the electropolymerizing comprises cyclic voltammetry, application of a constant potential, or combinations thereof. In some embodiments, the solvent comprises a phosphate buffered saline (PBS). The methods may involve adjusting a salt concentration of the PBS to adjust an electrical conductivity of the solvent for the electropolymerizing, and/or adding a salt (e.g., NaCl and/or KCl) to the PBS to control a permselectivity of the interference layer. In some embodiments, the glucose limiting layer comprises a polyurethane with a molecular weight greater than 100,000 Daltons that is physically crosslinked with a water-soluble polymer having a molecular weight greater than 100,000 Daltons. The water-soluble polymer of the glucose limiting layer may be polyacrylic acid, polyvinyl alcohol, polyvinylpyrrolidone, or poly(ethylene oxide). The polyurethane of the glucose limiting layer may be a thermoplastic silicone polyether polyurethane or a thermoplastic silicone polycarbonate polyurethane.

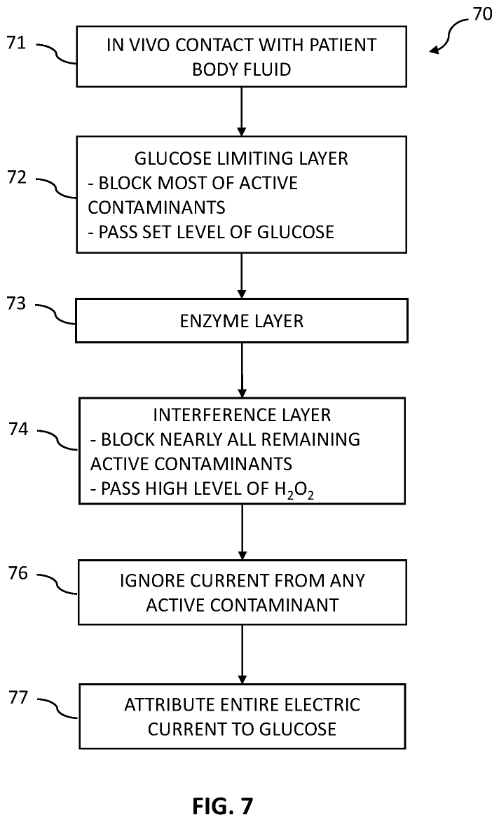

Referring now to FIG. 7, a flowchart for a method 70 for using a glucose-specific sensor is illustrated. The method may use, for example, the glucose-specific sensor 30 as described with reference to FIG. 3. In block 71, the glucose-specific sensor is inserted into a patient or user, where the working wire of the sensor makes contact with the patient's or user's body fluid such as blood or ISF. The body fluid will contain some level of glucose, as well as one or more active electrochemical contaminants such as acetaminophen, uric acid, or ascorbic acid. It will be appreciated that there are a wide variety of active electrochemical contaminants that may be in human blood, and that the levels and concentrations vary from individual to individual, and to a particular individual over time.

As shown in block 72, the body fluid contacts a glucose limiting layer that performs two key functions. First, it is set to pass a particular level of glucose from the body fluid into the enzyme layer, which increases linearity, and second, it is formulated and constructed to block most (e.g., over 95%) of the active electrochemical contaminants from ever reaching the enzyme layer. As discussed with reference to glucose-specific sensor 30, the glucose limiting layer is made from a high molecular weight (e.g., greater than 100,000 Daltons) polyurethane physically crosslinked with a high molecular weight (e.g., greater than 100,000 Daltons) water-soluble polymer. The high molecular weight polyurethane may be, for example, a thermoplastic polyurethane such as a thermoplastic silicone polyether polyurethane or a thermoplastic silicone polycarbonate polyurethane. Examples of water-soluble polymers that may be utilized to physically crosslink with the polyurethane include polyacrylic acid, polyvinyl alcohol, polyvinylpyrrolidone or poly(ethylene oxide) and other water-soluble polymers. This configuration for the GLL has been found, in accordance with the present disclosure, not only to substantially block the active electrochemical contaminants, but also passes sufficient glucose to support superior accuracy, sensitivity, and linearity.

An enzyme layer of block 73 is below the glucose limiting layer (i.e., between the glucose limiting layer and the interference layer) and receives the glucose, which is used with a GOx reaction to generate H₂O₂, which are attracted toward the electrically conducting substrate. The few active electrochemical contaminants that passed to the enzyme layer may also be attracted to the electrically conducting substrate. However, an interference layer of block 74 is placed between the enzyme layer and the conductive surface. This interference layer has two key features. First, it is applied very thin (e.g., 0.1 μm to 2.0 μm) so that the interference layer freely passes the H₂O₂ to the conductive surface. The interference layer is constructed to provide an exceptionally high electrical sensitivity of more than 1000 nA/mM. Second, the interference layer blocks the remaining active electrochemical contaminants. One skilled in the art would expect that such a thin and sensitive interference layer would provide little blocking of any of the active contaminants. However, by customizing the permeability of the interference layer (e.g., by adjusting a type of and/or an amount of salts), the interference layer is surprisingly able to reject or block nearly all of the active electrochemical contaminants that managed to pass into the enzyme layer. Thus, the interference layer performs two important but seemingly contradictory functions: easy passing of H₂O₂ while effectively blocking nearly all active electrochemical contaminants. The interference layer substantially blocks at least, for example, 80% or 90% or 95% of the contaminants that pass from the enzyme layer to the interference layer.

Block 76 shows that electric current generated from any active contaminant that has reached the conductive substrate can be ignored, which is a result of (1) the blocking of the bulk of the active contaminants by the glucose limiting layer and (2) the blocking of the few remaining active contaminants by the interference layer. These complementary effects result in a near zero generation of electrical signal due to any active contaminants in the patient's body fluid. Not only is the electric current from the active contaminants inconsequential, but due to the extreme electrical sensitivity of the interference layer, the electrical signal generated due to the H₂O₂ is very large. In this way, as shown in block 77, the electrical signal on the conductive substrate can be considered due to be only from the presence of glucose in the patient's body fluid, and the electrical effects of any active electrochemical contaminant can be fully ignored.

Reference has been made in detail to embodiments of the disclosed invention, one or more examples of which have been illustrated in the accompanying figures. Each example has been provided by way of explanation of the present technology, not as a limitation of the present technology. In fact, while the specification has been described in detail with respect to specific embodiments of the invention, it will be appreciated that those skilled in the art, upon attaining an understanding of the foregoing, may readily conceive of alterations to, variations of, and equivalents to these embodiments. For instance, features illustrated or described as part of one embodiment may be used with another embodiment to yield a still further embodiment. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only and is not intended to limit the invention.

What is claimed, is:

1. A glucose-specific sensor for in-vivo use in a patient, comprising:
a glucose limiting layer comprising a polyurethane with a molecular weight greater than 100,000 Daltons that is physically crosslinked with a water-soluble polymer having a molecular weight greater than 100,000 Daltons;
an enzyme layer comprising glucose oxidase (GOx) for reacting with in-vivo glucose in body fluid from the patient to generate hydrogen peroxide (H₂O₂);
an interference layer comprising a polymer formed from pyrrole, phenylenediamine (PDA), aminophenol, aniline, or combinations thereof, wherein the enzyme layer is between the interference layer and the glucose limiting layer; and
a substrate having a conductive surface adjacent the interference layer for carrying an electric current generated in response to an in-vivo glucose concentration of the patient.

2. The glucose-specific sensor of claim 1, wherein the body fluid is interstitial fluid (ISF).

3. The glucose-specific sensor of claim 1, wherein the water-soluble polymer of the glucose limiting layer comprises polyacrylic acid, polyvinyl alcohol, polyvinylpyrrolidone, poly(ethylene oxide), or combinations thereof to physically crosslink with the polyurethane.

4. The glucose-specific sensor of claim 1, wherein the polymer of the interference layer is electropolymerized on the substrate.

5. The glucose-specific sensor of claim 1, wherein the polymer of the interference layer is formed from a monomer and a co-monomer, the monomer being p-phenylenediamine.

6. The glucose-specific sensor of claim 5, wherein the co-monomer comprises 2-aminophenol, 3-aminophenol, 4-aminophenol, m-phenylenediamine, o-phenylenediamine, pyrrole, derivatized pyrrole, or the aniline.

7. The glucose-specific sensor of claim 1, wherein:
the body fluid in the patient further comprises active electrochemical contaminants;
the glucose limiting layer blocks greater than 95% of the active electrochemical contaminants from entering the enzyme layer; and
the interference layer substantially blocks the active electrochemical contaminants that have entered the enzyme layer from passing to the conductive surface.

8. The glucose-specific sensor of claim 1, wherein less than 1% of the generated electric current is due to electrochemical reactions of the active electrochemical contaminants.

9. The glucose-specific sensor of claim 1, wherein the generated electric current is less than 0.2 nA when the in-vivo glucose concentration is zero.

10. A glucose-specific sensor for in-vivo use in a patient, comprising:
a glucose limiting layer comprising a polyurethane with a molecular weight greater than 100,000 Daltons that is physically crosslinked with a water-soluble polymer;
an enzyme layer comprising glucose oxidase (GOx) for reacting with in-vivo glucose in body fluid from the patient to generate hydrogen peroxide (H₂O₂);
an interference layer comprising pyrrole and phenylenediamine (PDA), wherein the enzyme layer is between the interference layer and the glucose limiting layer; and
a substrate having a conductive surface adjacent the interference layer for carrying an electric current in response to an in-vivo glucose concentration of the patient.

11. The glucose-specific sensor of claim 10, wherein the body fluid is interstitial fluid (ISF).

12. The glucose-specific sensor of claim 10, wherein the water-soluble polymer has a molecular weight greater than 100,000 Daltons.

13. The glucose-specific sensor of claim 10, wherein the interference layer further comprises a co-monomer polymerized with the pyrrole and the PDA, the co-monomer being 2-aminophenol, 3-aminophenol, 4-aminophenol, m-phenylenediamine, o-phenylenediamine, p-phenylenediamine, or aniline.

14. The glucose-specific sensor of claim 10, wherein:
the body fluid in the patient further comprises active electrochemical contaminants;
the glucose limiting layer blocks greater than 95% of the active electrochemical contaminants from entering the enzyme layer; and
the interference layer substantially blocks the active electrochemical contaminants that have entered the enzyme layer from passing to the conductive surface.

15. The glucose-specific sensor of claim 10, wherein less than 1% of the generated electric current is due to electrochemical reactions of the active electrochemical contaminants.

16. The glucose-specific sensor of claim 10, wherein the generated electric current is less than 0.2 nA when the in-vivo glucose concentration is zero.
